(19) United States
(12) Patent Application Publication
(10) Pub. No.: US 2002/0012942 A1
(43) Pub. Date: Jan. 31, 2002

(54) POLYPEPTIDES WITH A4 INTEGRIN SUBUNIT RELATED ACTIVITY

(75) Inventors: JAMES B. MCCARTHY, MINNEAPOLIS, MN (US); LEO T. FURCHT, MINNEAPOLIS, MN (US); JOJI IIDA, SHOREVIEW, MN (US)

Correspondence Address: MERCHANT & GOULD PC, P.O. BOX 2903, MINNEAPOLIS, MN, 55402-0903, US

(73) Assignee: Regents of the University of Minnesota, Minneapolis, MN

(21) Appl. No.: 09/010,714

(22) Filed: Jan. 22, 1998

(51) Int. Cl. A61K 38/00; A61K 38/04; C07K 17/00; C07K 16/00; C07K 14/00; C07K 5/00; C07K 4/00; C07K 2/00; C07K 7/00; C12M 1/00; G01N 33/53; A61L 2/00; A61F 2/06; B05D 3/00

(52) U.S. Cl. 435/7.1; 514/12; 530/300; 530/328; 530/387.1; 435/283.1; 427/2.1; 623/1.1

(57) ABSTRACT

Polypeptides including an amino acid sequence corresponding substantially to KKEKDIMKK are provided. The polypeptides directly bind to MPG/NG2 proteoglycan and, when used as a soluble peptide, can inhibit melanoma cell adhesion. Antibodies generated against the polypeptides can bind α4β integrin and are capable of inhibiting α4β integrin-mediated cell adhesion. Medical applications such as prosthetic devices, percutaneous devices, cell culture substrates, affinity purification devices and chemodiagnostic devices are also provided.

### GOVERNMENT SUPPORT

This invention was made with government support under grant No. NCI 263 from the U.S. National Institutes of Health. The government has certain rights in the invention.

### BACKGROUND OF THE INVENTION

Integrins are a family of receptors that are fundamentally important for mediating cell adhesion to extracellular matrix (ECM) proteins. α4β1 integrin is important in tumor cell invasion and metastasis, although its exact role is complex and not completely understood. Tumor cells must adhere to variety of ECM proteins and molecules on other cells as they invade and metastasize. These interactions of tumor cells have a profound effect on their phenotype. α4β1 integrin is expressed on many hematopoietic malignancies and also on tumors such as melanomas. This integrin is unique among integrins in that it binds to both ECM components (e.g. fibronectin) and Ig superfamily adhesion receptors (e.g., VCAM-1) which are expressed on activated endothelial cells and other cell types. α4β1 integrin has been implicated in tumor cell arrest and/or extravasation, since agents which interfere with α4β1 integrin can inhibit experimental metastasis and pulmonary retention of injected tumor cells in mice tumor and/or cell/endothelial cell interactions in vitro. α4β1 integrin also binds to itself and promotes homotypic cell adhesion. Although a role for this integrin has clearly been established in modulating various aspects of tumor cell biology, the mechanisms by which the function of this integrin is modulated are complex and not well understood.

Cell surface proteoglycans are important in modulating cell adhesion and motility. Many cell adhesion promoting ECM components as well as certain adhesion promoting molecules expressed on the cell surface (e.g. CD31 or PECAM) can bind and support adhesion as a result of this interaction. Increasing evidence demonstrates that cell surface proteoglyeans act as a co-receptor of integrins and play a role in transducing signals distinct from signals from integrins. Cell surface proteoglycans interact with ECM proteins and often colocalize with integrins and/or stimulate integrin-mediated signaling through outside-in/inside-out mechanisms. For example, syndecan-4 localizes with α5β1 integrin in focal contacts and modulates focal contact formation by modulating the activation of protein kinase C in fibroblasts. It has recently been reported that cells transfected with syndecan-1 spread on substrata coated with fibronectin, while mock-transfectants did not. In addition, these transfectants adhered and spread on substrata coated with an anti-syndecan-1 core protein monoclonal antibody. These results suggest that the core protein of certain proteoglyeans (e.g. syndecan-1) can modulate integrin mediated cell spreading due to intracellular signaling events that are stimulated by the clustering of the core proteins.

Highly metastatic human A375SM melanoma cells express the vast majority of cell surface proteoglycans as chondroitin sulfate. Chondroitin sulfate protoglycan in melanoma cells was shown to modulate the activity of α4β integrin. Removal of cell surface chondroitin sulfate by treatment of cells with xyloside or chondroitinase ABC inhibits melanoma cell adhesion to the α4β1 integrin binding synthetic peptide CS1 (see, Iida et al., J. Cell Biol., 118 431-444 (1992)). CS1 is a fibronectin fragment corresponding to fibronectin amino acid residues 1961 to 1985 (the sequence numbering for fibronectin is as designated in U.S. Pat. No. 4,839,464) and has the sequence DELPQLVTLPHPNLHGPEILDVPST. The clustering of MPG/NG2 chondroitin sulfate protoglycan stimulated α4β1 integrin-mediated cell spreading and focal contact formation in melanoma cells has also been demonstrated, implicating a role for the MPG core protein in modulating integrin function by an inside-out signaling mechanism. In addition, cell surface chondroitin sulfate can modulate ligand binding by α4β1 integrin, suggesting that it may bind to, and directly modulate, the ligand properties of the integrin. Thus, the cell surface MPG/NG2, chondroitin sulfate protoglycan, can modulate the function of α4β1 integrin by a complex mechanism involving intracellular signals and perhaps by conformational influences on the extracellular domain of the integrin.

α4β1 integrin can assume multiple activation states as with other integrins. Various monoclonal antibodies, divalent cations, or phorbolmyristate acetate (PMA) can activate α4β1 integrin. Distinct conformational changes associated with α4β1 integrin activation or ligand binding have been identified. α4β1 integrin is cleaved into 70 kD and 80 kD fragments on certain activated lymphocytes, although the cleavage does not affect the integrin function. Other studies demonstrated that residues 108-268 encompass putative ligand binding sites, in which bivalent-cation-binding motif is not included. Recently, it has been demonstrated that mutation of Cys₇₁₇, which localizes close to the plasma membrane, disrupts integrin function presumably as a result of long range influences of Cys₇₁₇ on the conformation of the ligand binding site. Point mutation of Asp₆₉₈ to Ala resulted in the dysfunction of α4β1 integrin, while the mutant integrin could still interact with ligand by affinity chromatography, suggesting that the failure for supporting cell adhesion might be related with the integrin clustering or signaling.

Collectively, regulation of integrin functions on cell surface have been studied to understand the molecular mechanisms of, inflammation and tumor metastasis. In this regard, it is important to realize that cell surface proteoglycans can act not only as a cell surface receptor but also as a important modulator of integrins. The fact that proteoglycan binding sites are often expressed in close proximity to integrin binding domains within ECM proteins or cell surface adhesion molecules suggests that cellular recognition of the ECM or counter-adhesion receptors on opposing cells might involve the formation of a receptor cluster on the plasma membrane that includes both proteoglycans and integrins. Understanding the nature of such interactions may help to explain cell-type specific behavior on ECM proteins that are often observed for integrins. There is, accordingly, a continuing need to identify peptides derived from α4 integrin subunits that are capable of modulating cell adhesion.

### SUMMARY OF THE INVENTION

The present invention provides provides which are related to a fragment of the α4 integrin subunit. The polypeptides can be prepared by conventional solid phase peptide synthesis or through standard recombinant methods of peptide production.

The present invention relates to polypeptides which typically include an amino acid sequence of at least about eight amino acids and, preferably, at least about twelve amino acids, corresponding substantially to the sequence of a fragment from the α4 integrin subunit. While the present method is illustrated herein with α4 itegrin subunit related polypeptides which include at least ten amino acid, it is to be understood that polypeptides having shorter α4 itegrin subunit related sequences of amino acids are within the scope of the invention. For example, polypeptides having sequences of about 2 or 3 amino acids or larger which retain the α4β integrin-mediated cell adhesion inhibition functional activity are within the scope of the invention. Examples of polypeptide fragments of this size which exhibit the functional activity observed with a larger reltated peptide have been reported. Examples of such polypeptides include arg-gly-asp (RGD), arg-gly-asp-ser (RGDS) and other RGD-containing tetramers. It is known that trimers such as RGD when connected to other amino acids or amino acid sequences are functionally active (e.g., the Ser of RGDS may be substituted to form a functionally active tetramer). Other examples of functionally active small polypeptides include ala-arg-ile (ARI), arg-ala-arg-ile (RARI) and other short ARI-containing sequences.

The polypeptides are capable of modulating cell adhesion and/or cell migration.

The polypeptides generally have a net charge of at least +3 and/or a net hydropathy of about −10 to abou −20 and include a close grouping of both positively and negatively charged residues. The polypeptides of the present invention preferably are capable of inhibiting adhesion of cells (e.g., melanoma cells or Ramos cells) to fibronectin or the fibronectin fragment CS1 and/or are capable of binding to MPD/MG2 proteoglycan.

The polypeptides of the invention modulate cell adhesion and are related to particular fragments of the α4 itegrin subunit. Preferred polypeptides of the invention include the amino acid sequence KKEKDIMKK (SEQ ID NO:1) or a closely related variant of this amino acid sequence. The KKEKDIMKK amino acid sequence corresponds to residues 573-581 of the α4 integrin subunit. Suitable examples of such polypeptides include polypeptides containing the amino acid sequence LQQKKEKDIMKKTI (SEQ ID NO:2). The present polypeptides may be in free form or may be covalently bound to a carrier molecule (such as ovalbumin) as part of a polypeptide/carrier molecule conjugate which includes one or more of the present polypeptides. In another embodiment, the polypeptide may be covalently incorporated into an amphiphilic molecule (see, e.g., Berndt et al., J. Am. Chem. Soc., 117, 9515-9522 (1995), the disclosure of which is incorporated herein by reference). Suitable amphiphilic molecules typically include at least one fatty acyl or alkyl group as well as one of the present polypeptides. Lipid-like amphiphilic molecules of this type are typically capable of being formed into a microscopic structure, such as a liposome.

The present invention also provides an affinity substrate (e.g., beads formed from a synthetic resin) having a surface coated with at least one of the polypeptides of the invention (or a conjugate thereof). The affinity substrate is capable of selectively binding MPG/NG2 cell surface proteoglycan. The substrate may be a surface which includes a synthetic resin, a portion of a bead, a portion of a macroporous fiber, or the wells of a microtiter plate.

The present invention also provides a peptide comprising a F_{ab} fragment from an antibody specific for an antigen within an amino acid sequence including KKEKDIMKK (SEQ ID NO:1) or a variant thereof. The variant has at least 9 amino acid residues and differs from KKEKDIMKK (SEQ ID NO:1) through one or more deletions, conservative amino acid substitutions or combinations thereof. Typically the F_{ab} fragment is from an antibody specific for an antigen within an amino acid sequence corresponding to LQQKKEKDIMKKTI (SEQ ID NO:2). One example of such antibodies is an IgG antibody specific for a polypeptide having the amino acid sequence LQQKKEKDIMKKTI (SEQ ID NO:2).

### BRIEF DESCRIPTION OF THE DRAWINGS

FIG. 1 depicts the location of several synthetic polypeptides derived from α4 integrin.

FIG. 2 shows the relative adhesion of A375SM human melanoma cells to substrates coated with CS1/ovalbumin conjugates (“CS1/OVA”) in the presence of various synthetic α4 integrin subunit fragments. Inhibition by the fibronectin fragment CS1 was employed as a positive control.

FIG. 3 shows the relative adhesion of A375SM human melanoma cells to substrates coated with CS1/ovalbumin conjugates in the presence of L-SG1, D-SG1 or CS1.

FIG. 4 shows the effect of antibodies to various α4 integrin fragments on the adhesion of A375SM human melanoma cells to substrates coated with CS1/ovalbumin conjugates.

FIG. 5 shows the effect of anti-SGl and normal rabbit IgG antibodies and Fab fragments on the adhesion of A375SM human melanoma cells to substrates coated with CS1/ovalbumin conjugates.

FIG. 6 shows the effect of anti-SG1 IgG and normal rabbit IgG antibodies on the adhesion of Ramos cells to substrates coated with CS1/ovalbumin conjugates.

FIG. 7 shows the profile of MPG/NG2 proteoglycan eluted with a linear gradient of sodium chloride from an affinity column containing SG1 polypeptide immobilized on CH-affinity beads. Open circles represent radioactivity in each fraction and the solid line represents the concentration of NaCl in each fraction.

FIG. 8 shows the profile of radiolabeled MPG/NG2 proteoglycan eluted with a linear gradient of sodium chloride from an affinity column containing SG1 polypeptide immobilized on CH-affinity beads. Open circles represent radioactivity in each fraction and the solid line represents the concentration of NaCl in each fraction.

FIG. 9 is a graph showing the adhesion of melanoma (A375SM) cells to Immulon I wells coated with an SG1-ovalbumin conjugate. The A375SM cell adhesion observed with ovalbumin coated wells is shown as a negative control.

### DETAILED DESCRIPTION OF THE INVENTION

Cellular recognition of the ECM proteins and of other cells has a complex molecular basis, involving multiple distinct cell surface receptors. The results reported herein demonstrate that the potential chondroitin sulfate binding site in the α4 integrin subunit plays a role in α4β1 integrin-mediated melanoma cell adhesion. The involvement of the SG1 site in the context of α4β1 function was further demonstrated by the results in which anti-SG1 IgG as well as its Fab fragments inhibit cell adhesion to CS1. The data also demonstrate that MPG/NG2 directly interacts with the polypeptide SG1 in affinity chromatography. These results collectively show that α4β1 integrin may interact with MPG/NG2 through SG1 and the interaction is required for maintaining the function of the integrin.

Glycosaminoglycans have been implicated in modulation of integrin function although the exact mechanism is not known. It has been demonstrated that heparan sulfate plays a role in formation of focal contacts in fibroblasts. It has also been reported that cell adhesion to basement membrane proteoglycan, perlecan, is influenced by the heparan sulfate glycosaminoglycans on the core protein. Recent results showed that I-domain of CD 11b/CD 18 can directly interact with heparin and/or heparan sulfate glycosaminoglycan chains which are expressed on endothelial cells. Removing chondroitin sulfate is known to reduce α4β1 integrin-mediated melanoma cell adhesion and ligand binding. Importantly, the reduced α4β1 integrin function can be restored by an activation monoclonal antibody anti-β1 (8A2). These results show that glycosaminoglycan chains can modulate the conformation of the integrin, possibly through direct interaction between the two distinct cell surface receptors.

Polypeptide SG1 is located to the carboxy-terminal side of the protease cleavage site within the α4 integrin subunit. Thus, it is located well outside the β1 ligand binding sites within the α4 integrin subunit. Although the details of the mechanisms of the potential interaction between MPG/NG2 and α4β1 integrin are not well understood, the results described herein suggest that the interaction of the two distinct cell surface receptors through the SG1 site may affect adhesive functions of α4β1 integrin by long range structural influences on one or more ligand binding sites of the integrin.

For the purposes of the invention, the definition of the polypeptides corresponding substantially to an amino acid sequence within the α4 integrin subunit includes peptides which correspond to an amino acid sequence within an allelic variant or mutant of this integrin subunit. The variants and mutants possess a high degree of sequence homology with the native sequence (e.g., substitition, deletion or addition mutants). Polypeptides which correspond substantially to the amino acid sequence of a SG1 fragment typically have a high degree of homology (e.g., at least about 90% homology) with the SG1 fragment sequence. Preferably, the present polypeptides have at least about 80% and more preferably at least about 90% sequence identity with the native sequence.

In addition to corresponding substantially to an amino acid sequence within the α4 integrin subunit, the present polypeptides retain one or more desirable properties of the α4 integrin subunit. For example, the polypeptides maintain the functional activity of the α4 integrin subunit to modulate cell adhesion, spreading, motility, and/or ligand binding. For example, polypeptides of the invention can modulate adhesion of melanoma cells and Ramos cells to proteins such as fibronectin and/or to a substrate including these proteins on its surface (e.g., to the surface of an inanimate support such as a well of a microtiter plate). Preferably, the polypeptides are capable of inhibiting the adhesion of melanoma cells or Ramos cells to a CS1-coated substrate.

Preferably, the present variants of α4 integrin fragments are modified through deletions or conservative amino acid substitutions. Typically, such conservative amino acid substitutions include substitutions such as described by Dayhoff in the “Atlas of Protein Sequence and Structure,” 5, (1978) and Argos in EMBO J., 8, 779 (1989), the disclosures of which are herein incorporated by reference. For example, the exchange of amino acids within one of the following classes represent conservative substitutions: Class I: Ala, Gly, Ser, Thr, and Pro (representing small aliphatic side chains and hydroxyl group side chains); Class II: Cys, Ser, Thr and Tyr (side chains including an —OH or —SH group); Class III: Glu, Asp, Asn and Gln (representing carboxyl group containing side chains): Class IV: His, Arg and Lys (representing basic side chains); Class V: Ile, Val, Leu, Phe and Met (representing hydrophobic side chains); Class VI: Phe, Trp, Tyr and His (representing aromatic side chains); and Class VII: Lys, Asp, Glu, Asn and Gln. The classes also include related amino acids such as 3Hyp and 4Hyp in Class I; homocysteine in Class II; 2-aminoadipic acid, 2-aminopimelic acid, γ-carboxyglutamic acid, β-carboxyaspartic acid, and the corresponding amino acid amides in Class III; ornithine, homoarginine, N-methyl lysine, dimethyl lysine, trimethyl lysine, 2,3-diaminopropionic acid, 2,4-diaminobutyric acid, homoarginine, sarcosine and hydroxylysine in Class IV; substituted phenylalanines, norleucine, norvaline, 2-aminooctanoic acid, 2-aminoheptanoic acid, statine and β-valine in Class V; and naphthylalanines, substituted phenylalanines, tetrahydroisoquinoline-3-carboxylic acid, and halogenated tyrosines in Class VI.

Larger compilations of related amino acids and amino acid derivatives may be found in a variety of publications known to those skilled in the art, e.g., the catalogue of Bachem Biosciences, Inc. (King of Prussia, Pa.). Moreover, the classes may include both L and D stereoisomers, although L-amino acids are typically preferred for substitutions. As used herein, the term “conservative amino acid substitutions” also includes a number of other amino acid substitutions identified as frequently occurring conservative amino acid substitutions by Gribskov et al., Nucl. Acid Res., 14(16), 6745 (1986), the disclosure of which is herein incorporated by reference. Included among such conservative amino acid substitutions are the exchange of Ala with Cys, Asp or Glu; the exchange of Gly or His with Asp, Glu or Gln; the exchange of Ser with Asn, Phe or Trp; the exchange of Leu with Tyr or Trp; and the exchange of Pro with Glu, Gln or Arg.

Preferred α4 integrin subunit fragments and their variants have a calculated or actual net charge at neutral pH of +3 and/or a net hydropathy of −10 to −20. The net charge of a polypeptide is the sum of all charged residues where lysine and arginine residues have a single positive charge and glutamate and aspartate residues have a single negative charge at neutral pH. Histidine is assumed to be uncharged at neutral pH. In some polypeptide sequences, the sequence may alter the actual pKas of amino acid side chains and result in an actual net charge that varies from the calculated net charge. Net hydropathy relates to the hydrophilicity of a peptide such that a hydrophilic region of a protein has a negative hydropathy index. Hydropathy indexes can be calculated according to the methods of Kyte et al., J. Mol. Biol. 157, 105-132 (1982).

Exemplary α4 integrin subunit fragments of the present invention include:

SG1—LQQKKEKDIMKKTI (SEQ ID NO:2)

A4-105—KYKAFLDKQNQVKFG (SEQ ID NO:3)

The SG1 polypeptide (SEQ ID NO:2) corresponds to residues 570-583 from the sequence of human α4 integrin subunit, has a net charge of +3, and a net hydropathy of −14.4. The A4-105 peptide (SEQ ID NO:3) corresponds to residues 201-215 of the human α4 integrin subunit, has a net charge of +3, and a net hydropathy of −14.7. The residue numbers from the human α4 integrin subunit are as reported by Takada et al., EMBO. J., 8, 1361-1368 (1989).

Variants of the SG1 polypeptide include may include truncated variants, variations with amino acid substitutions, and/or variations with deleted amino acids. The variants typically correspond substantially to a fragment of the native human α4 integrin subunit sequence. Preferably, the variants are highly homologous with the native sequence and have at least about 70% and more preferably at least about 90% identity with a fragment of the native sequence. Most preferably, the variants only differ from the native sequence by one or more conservative amino acid substitutions.

One example of a suitable truncated variant is the “SG1a” polypeptide. The SG1a amino acid sequence corresponds to residues 573-582 of the human α4 integrin subunit and has the sequence KKEKDIMKKT (SEQ ID NO:4). The SG1a polypeptide has a net charge of +3 and a net hydropathy of −14.4.

Preferred variants of the present polypeptide include the following structural motif or a closely related sequence:

KKEKDIMKK (SEQ ID NO: 1).

Variations of this structural motif may include motifs in which one or more, preferably one, of the lysines is substituted with a positively charged amino acid, preferably arginine. Preferred variations on this motif can also include substitutions of a negatively charged amino acid (e.g., an amino acid residue having a side chain carboxylic acid group) for a glutamate and/or aspartate residues in the motif. Preferably, only one of the negatively charged amino acids is substituted, and more preferably is substituted with either glutamate or aspartate. Another preferred variation on the structural motif is substitution for or deletion of one or both of the hydrophobic amino acids isoleucine and methionine. These hydrophobic amino acids are substituted with another hydrophobic amino acid, preferably isoleucine, valine, leucine, phenylalanine, or methionine. This structural motif can be included in a longer polypeptide, e.g., a polypeptide having no more than about 100 amino acid residues, preferably no more than about 50 and, more preferably, no more than about 25 amino acid residues. For example, the longer polypeptide may have 1-5 amino acids attached to either the N-terminal and/or C-terminal end of the structural motif. These additional amino acids typically have a small aliphatic side chain, a hydrophobic side chain, or a side chain with a hydrogen bonding group such as an hydroxyl group, a sulfhydryl group, or an amide.

Synthesis of Polypeptides

The polypeptides of the invention may be synthesized by the solid phase method using standard methods based on either t-butyloxycarbonyl (BOC) or 9-fluorenylmethoxy-carbonyl (FMOC) protecting groups. This methodology is described by G. B. Fields et al. in Synthetic Peptides: A User's Guide, W. M. Freeman & Company, New York, N.Y., pp. 77-183 (1992). Polypeptide structures and purity can be analyzed by HPLC, and amino acid analysis and sequencing. The polypeptides as described in the examples herein were synthesized to correspond to sequences of the α4 integrin subunit.

The present peptides may also be synthesized via recombinant techniques well known to those skilled in the art. For example, U.S. Pat. No. 5,595,887, the disclosure of which is herein incorporated by reference, describes methods of forming a variety of relatively small peptides through expression of a recombinant gene construct coding for a fusion protein which includes a binding protein and one or more copies of the desired target peptide. After expression, the fusion protein is isolated and cleaved using chemical and/or enzymatic methods to produce the desired target peptide.

Polypeptide Carrier Conjugates

The polypeptides of the present invention may be employed in a monovalent state (i.e., free polypeptide or single polypeptide fragment coupled to a carrier). Often, the polypeptides are employed as conjugates of multiple polypeptide fragments bound to a carrier. The carrier may be a biological carrier molecule, such as an albumin, collagen or a synthetic polymer (e.g., a polymer of the type typically used as a chromatography support). Examples of suitable carrier molecules include ovalbumin, human serum albumin, other proteins, synthetic polymers such as polyethylene glycol. Modification of the present polypeptides by attachment to a carrier molecule may increase the apparent affinity or change the stability of a polypeptide. The number of polypeptide fragments associated with or bound to each carrier molecule can be varied, but from about 4 to 8 polypeptide fragments per carrier molecule are typically obtained under standard coupling conditions.

For instance, polypeptide/carrier molecule conjugates may be prepared by treating a mixture of polypeptides and carrier molecules with a coupling agent, such as a carbodiimide. The coupling agent may activate a carboxyl group on either the polypeptide or the carrier molecule so that the carboxyl group can react with a nucleophile (e.g., an amino or hydroxyl group) on the other member of the polypeptide/carrier molecule, resulting in the covalent linkage of the polypeptide and the carrier molecule.

For example, conjugates of a polypeptide coupled to ovalbumin may be prepared by dissolving roughly equal amounts of lyophilized polypeptide and ovalbumin in a small volume of water. In a second tube, l-ethyl-3-(3-dimethylamino-propyl)-carboiimide hydrochloride (EDC; ten times the amount of polypeptide) is dissolved in a small amount of water. The EDC solution was added to the polypeptide/ovalbumin mixture and allowed to react for a number of hours. The mixture may then dialyzed (e.g., into phosphate buffered saline) to obtain a purified solution of polypeptide/ovalbumin conjugate. Polypeptide/carrier molecule conjugates prepared by this method typically contain about 4 to 5 polypeptide fragments per ovalbumin molecule.

A polypeptide of the invention may be coupled to a chromatography support by methods known to those of skill in the art. Frequently, a manufacturer of the chromatography support provides instructions and reagents for linking peptides to their particular supports. Polypeptides of the invention can be linked to materials, such as glass, ovalbumin, agarose, synthetic resins, long-chain polysaccharides, or dextran-based microspheres coated with denatured collagen using coupling reagents such as EDC to form affinity chromatography materials. Such chromatography materials can be used to isolate or purify antibodies recognizing polypeptides in the invention, cells binding to or recognizing peptides of the invention, and other proteins or macromolecules that interact with the polypeptides of the invention.

Lipid-like Amphiphilic Derivatives

The polypeptides of the present invention may also be incorporated into an amphiphilic molecule. As employed herein, the term “amphiphilic molecule” refers to a molecule having a water soluble polar portion (the polypeptide portion) and a water-insoluble organic portion. Typically, the amphiphilic molecule is a lipid-like amphiphilic molecule which includes two long fatty chains (e.g., two fatty acyl or alkyl chains having about 10 to about 20 carbon atoms) and one of the present polypeptides covalently bonded to a linking segment. Suitable linking segment include fragments derived from an aminocarboxylic acid (e.g., aminodicarboxylic acid such as glutamic acid or aminosuccinic acid) or an aminoalcohol (e.g., an aminoalkanediol such as 2-amino-1,3-propanediol). The amphiphilic molecules are typically capable of forming stable monolayers at an air-water interface and/or associating to form other molecular assemblies such as bilayer membrane systems, micelles and/or vesicles.

Examples of such amphiphilic molecules are described in Yu et al., J. Am. Chem. Soc., 118, 12515-12520 (1996) and Berndt et al., J. Am. Chem. Soc., 117, 9515-9522 (1995), the disclosure of which are incorporated herein by reference. The polypeptide amphiphiles described in the Yu and Berndt references include a fatty alcohol diester of glutamic acid covalently bonded to a polypeptide through a succinic acid diamide linking the N-terminus of the polypeptide and the α-amino group of the glutamate moiety. Alternatively, the polypeptide could be linked to the α-amino group of the glutamate moiety through an amide bond with the C-terninal α-carboxylate group of the polypeptide. Other alternative embodiments of amphiphilic molecules of this type include the polypetide covalently covalently bonded to the linking segment through a amide bond to a side chain amino or carboxylate group. Other examples of suitable lipid-like amphiphilic molecules include compounds having the polypeptide covalently linked through an amide bond to the amino group of a fatty acid diester of an aminoalkanediol (e.g., aminopropanediol) or to the amino group of a phosphatidyl-ethanolamine.

Practical Applications

The results described herein indicate that the present α4 integrin subunit-related polypeptides are capable of modulating the process of cell adhesion. A number of the practical applications for the polypeptides of the present invention can be envisioned. Such applications include the promotion of the healing of wounds caused by the placement of synthetic substrata within the body. Such synthetic substrata can include artificial vessels, intraocular contact lenses, hip replacement implants and the like, where cell adhesion is an important factor in the acceptance of the synthetic implant by normal host tissue.

As described in U.S. Pat. No. 4,578,079, medical devices can be designed making use of these polypeptides to attract cells to the surface in vivo or even to promote the growing of a desired cell type on a particular surface prior to grafting. An example of such an approach is the induction of endothelial cell growth on a prosthetic device such as a blood vessel, heart valve or vascular graft. Such a device is generally woven or knitted from nitrocellulose or polyester fiber, particularly Dacron TM (polyethylene terephthalate) fiber. A variety of cells are attracted to type I collagen and to the present polypeptides. The latter point indicates the potential usefulness of these defined polypeptides in coating a patch graft or the like for aiding wound closure and healing following an accident or surgery. The coating and implantation of synthetic polymers may also assist in the regeneration of nerves following crush traumas, e.g., spinal cord injuries.

In such cases, it may be advantageous to couple the peptide to a biological molecule, such as albumin, a glycosaminoglycan or a proteoglycan. It is also indicative of their value in coating surfaces of a prosthetic device which is intended to serve as a temporary or semipermanent entry into the body, e.g., into a blood vessel or into the peritoneal cavity, sometimes referred to as a percutaneous device. Such devices include controlled drug delivery reservoirs or infusion pumps.

Also, the polypeptides of the present invention can be used to promote cell adhesion of various cell types to a naturally occurring or artificial substrate intended for use in vitro. For example, a culture substrate such as the wells of a microtiter plate or the medium contacting surface of microporous fibers or beads, can be coated with the cell-attachment polypeptides. This can obviate the use of type I collagen in the medium, thus providing better defined conditions for the culture as well as better reproducibility.

One example of commercial cell attachment surfaces are Cytodex 3® microcarriers (manufactured by Pharmacia), which are dextran-based microspheres coated with denatured collagen. The Cytodex 3® microcarriers make it possible to grow the same number of adherent cells in a much smaller volume of growth medium than would be possible in a dish. The activity of these beads is generally dependent on the use of coating protein in the growth medium. The α4 integrin subunit-related polypeptides are expected to provide an improved, chemically-defined coating which could be employed for such purposes. Other surfaces or materials, such as glass, agarose, synthetic resins or long-chain polysaccharides, may also be coated to enhance their ability to serve as cell attachment sites.

The invention will be further described by reference to the following detailed examples. The examples are meant to provide illustration and should not be construed as limiting the scope of the present invention.

### EXAMPLES

Cell Culture:

Highly metastatic human melanoma cells, A375SM, which were selected by in vivo experimental metastasis assays of parent A375P cells in nude mice were kindly provided by Dr. I. J Fidler (M. D. Anderson Hospital Cancer Center, Houston, Tex.). The cells were maintained in Eagle's Minimum Essential Medium (“EMEM”) supplemented with 10% fetal calf serum, 50 ug/ml gentamycin, vitamin solution, and 1 mM sodium pyruvate. Ramos cells were provided by Dr. Tucker Lebien (Laboratory Medicine and Pathology, University of Minnesota, Minn.) and maintained RPMI1640 supplemented with 10% fetal calf. These cells were routinely used after less than 15 passages from frozen stocks in order to minimize phenotypic drift.

Monoclonal Antibodies (mAbs):

P4C2 (anti-α4), PlD6 (anti-α5), and P5D2 (anti-β1) were provided by Dr. Elizabeth A. Wayner (University of Washington, Wash.). The specificities of these antibodies have been previously demonstrated. Anti-NG2 core protein antibody (9.2.27) was provided by Dr. Ralf Reisfeld (The Scrips Research Institute, San Diego, Calif.; Bumol et al., Proc. Natl. Acad. Sci., 79,1245-1249 (1982)). Unique glycoprotein-proteoglycan complex defined by monoclonal antibody on human melanoma cells. Proc. Natl. Acad. Sci. 79:1245-1249. Anti-CD44 mAb (clone P1G12) was purchased from Telios Pharmaceuticals, Inc. CA).

Protein Isolation:

Human plasma fibronectin was purified as a by-product of factor VIII production by sequential ion-exchange and gelatin affinity chromatography as described in McCarthy et al., J. National Cancer Institute, 80, 108-116 (1988). Metastasis inhibition of different tumor types by pruified laminin fragments and a heparin binding fragment of fibronectin (McCarthy et al., J. National Cancer Institute 80, 108-116 (1988)). The trypic/catheptic 33 kD heparin binding fragment of fibronectin A-chains was purified according to methods previously reported. Purity of fibronectin or a trypic, RGD-containing 75 kD fragment was verified by SDS-PAGE and Coomassie Brilliant Blue staining.

Polypeptide Synthesis and Characterization:

Polypeptides used in these examples were synthesized at the Microchemical Facility of the University of Minnesota (Minneapolis, Minn.) using a Beckman System 990 peptide synthesizer. The procedures used were based on the Merrifield solid phase system as described previously in lida et al., Cancer Res., 55, 2177-2185 (1995)). Spreading and focal contact formation of human melanoma cells in response to the stimulation of both melanoma-associated proteoglycan (NG2) and α4β1 integrin (lida et al., Cancer Res., 55, 2177-2185 (1995)). Lyophilized crude peptides were purified by preparative reverse-phase HPLC on a C-18 column, using an elution gradient of 0-60% acetonitrile with 0.1% trifluoroacetic acid in water. The purity and composition of the peptides were verified by HPLC analysis of hydrolysates prepared by treating the peptides under nitrogen in 6N HCl overnight at

Generation and Purification of Polyclonal Antibodies.

Synthetic polypeptides were coupled to keyhole limplet hemocyanin (KLH, Sigma Chemical Co., St. Louis, Mo.) using 1-ethyl-3(3-dimethylaminopropyl)-carbodiimide hydrochloride (“EDC;” Sigma Chemical Co.) as a coupling reagent. The conjugate was mixed with Freund's adjuvant and then used to immunize New Zealand White Rabbits. IgG was purified from pooled immune sera by precipitation with ammonium sulfate followed by DEAE anion exchange chromatography. Purity of the IgG was determined by SDS-PAGE and Coomassie Brilliant Blue staining of the gels. Specificity of the purified IgG was then determined by ELISA. Fab fragments of the anti SG1 IgG or normal rabbit IgG were obtained by incubating the purified IgG with immobilized papain (Pierce Chemical Co., Rockford, Ill.) for 5 hr at 37° C. following the manufacturer's instructions. The digestion products were examined by SDS-PAGE and were found to be free of intact IgG heavy chain.

Conjugation of CS1 Polypeptide to Ovalbumin:

CS1 polypeptide was chemically conjugated to ovalbumin (“OVA”) using EDC, since previous studies have shown that the coupling of synthetic polypeptides to larger carrier proteins resulted in enhanced cell adhesion activities. Equal amounts (by weight) of peptide CS1 and OVA were solubilized in water and mixed with a 10 fold excess (by weight) of EDC dissolved in water. The sample was then mixed overnight at 4° C. on a circular rotator. The coupled peptide was then dialyzed extensively against PBS to remove the excess EDC and uncoupled peptide (Spectrapore 6, 10 kD exclusion, Spectrum Medical Industries, Los Angeles, Calif.). The conjugates were stored at −20° C. until use. The specificity of cell adhesion to CS1/OVA was verified by using mAbs against α4 and β1 as well as soluble peptide CS1 as described.

Cell Adhesion Assays:

Cell adhesion assays were performed as previously described in Iida et al., J. Cell Biol., 118, 431-444 (1992) with minor modifications. Ligands were diluted in PBS, and 50 μl aliquots were dispersed in triplicate into Immulon-1 polystyrene microtiter wells. The wells were incubated at 37° C. overnight, and nonspecific binding sites on plastic were then blocked by treating the wells with 200 ul of PBS/BSA.

Subconfluent A375SM human melanoma cells which had been radiolabeled overnight with ³H-thymidine (3HTdR, specific activity 6.7 Ci/mmol; NEN Research Products, Boston, Mass.), were harvested by rinsing with 1 mM EDTA, washed two times with EMEM/BSA (EMEM containing 2 mg/ml BSA and 0.15 mM Hepes, pH 7.2) and adjusted to a concentration of 1 05 cells/ml in the same medium. Aliquots of 100 ul of the cell suspension were dispensed into the wells and the cells were incubated at 37° C. for 30 min. The assays were terminated by aspirating loosely bound and unbound cells from the wells, washing the wells three times, and solubilizing the bound cells in 0.5 N NaOH containing 1% SDS. Bound radioactivity, determined in a Beckman Model 3801 Liquid Scintillation Counter, was used to calculate the percentage of cells which remained adherent to each substratum. Unless otherwise indicated, the data represent the mean of triplicate determinations.

Immunoprecipitations:

Ramos cells were extracted with 50 mM Tris-HCl (pH 7.3) containing 50 mM b-octylglucoside, 15 mM NaCl, 1 mM Ca²⁺, 1 mM Mg²⁺, 1 mM Mn²⁺, 1 mM PMSF, and 1 mM NEM (extraction buffer) and then centrifuged at 36,500 rpm for 1 h at 4° C. The supernatants were precleared and then immunoprecipitated with n-mAbs anti-a4, anti-pi, or control IgG. The beads were washed with 50 mM Tris-HCl (pH 7.3) containing 50 mM B octylglucoside, 0.1% Tween-20, 0.4 M NaCl, 1 mM Ca²⁺, 1 mM Mg²⁺, 1 mM Mn²⁺, 1 mM PMSF, and 1 mM NEM.

SDS-PAGE and Western Blotting:

The immunoprecipitated proteins were separated by 7.5% running with 5% stacking gels and then transferred onto Immobilon-P membrane. Nonspecific binding sites on the membranes were blocked with PBS containing 3% BSA overnight at 4° C. The membranes were then incubated with biotineated-rabbit IgG for 4 hr at room temperature followed by an incubation with peroxidase-conjugated streptoavidine for 1 hr at room temperature. Immunoreactivity was visualized by using an enhanced chemiluminescence systems (Boeringer-Mannheim, Germany).

Affinity Chromatography:

Peptide-affinity chromatography was performed according to the methods described in Iida et al., J. Cell Biol., 118, 431-444 (1992). SG1 was immobilized on CH-Spharose and blocked using standard methods as described in the manufacturer's instruction. (available from Pharmacia). Human melanoma cells were surface radiolabeled with ¹²⁵I using lactoperoxidase and extracted as described in Iida et al., Cancer Res., 55, 2177-2185 (1995). Cells were extracted with 50 mM Tris-HCl (pH 7.3) containing 50 mM β-octylglucoside, 15 mM NaCl, 1 mM Ca²+, 1 mM Mg²+, 1 mM Mn²+, 1 mM PMSF, and 1 mM NEM (extraction buffer) and then centrifuged at 36500 rpm for 1 h at 4° C. The cell lysates were first precleared by incubating with a mock-column, where no peptide was immobilized, and then applied on the SG1 column. The column was extensively washed with the extraction buffer and then bound proteins were subsequently eluted by a linear gradient of sodium chloride (15 mM to 1 M). Each fraction was counted by r-counter and positive fractions were pooled and immunoprecipitated as described below.

The sample was first precleared with normal mouse IgG/rabbit anti-mouse IgG/protein A-agarose for 2 h at 4° C. 1 ug of 9.2.27 was incubated with rabbit anti-mouse IgG/protein A-agarose for 4 h at 4° C. The equal counts of the precleared cell lysates were incubated with the 9.2.27/rabbit anti-mouse IgG/protein A-agarose for 4 to 6 h at 4° C. The immune complexes were extensively washed with 50 mM Tris-HCl (pH 7.3) containing 50 mM β-octylglucoside, 0.1% Tween-20, 0.4 M NaCl, 1 mM Ca²⁺, 1 mM Mg²⁺, 1 mM Mn²⁺, 1 mM PMSF, and 1 mM NEM. For enzyme treatment, the beads were washed two times with the digestion buffer (0.1 M Tris-HCl, pH 8.0 containing 30 mM sodium acetate, 10 mM EDTA, 10 mM NEM, 5 mM PMSF, and 0.36 mM pepstatin) and then incubated with 0.01 U/ml of protease-free chondroitinase ABC (Seikagaku America, Inc. Rockville, Md.) in the digestion buffer for 4 h at 37° C. For heparitinase treatment, digestion buffer (0.1M Tris-HCl, pH7.2 containing 5 mM sodium acetate, 0.01M EDTA, 0.01M NEM, 1 mM PMSF, 0.36 mM pepstatin) was used with 10 mU of heparitinase (Seikagaku America). The immune complexes were released from the beads with boiling in SDS-sample buffer and then analyzed on SDS-PAGE (7.5% running gel with 5% stacking gel). The gels were dried and exposed on X-ray films at −80° C.

### Example 1

Inhibition of Melanoma Adhesion to CS1

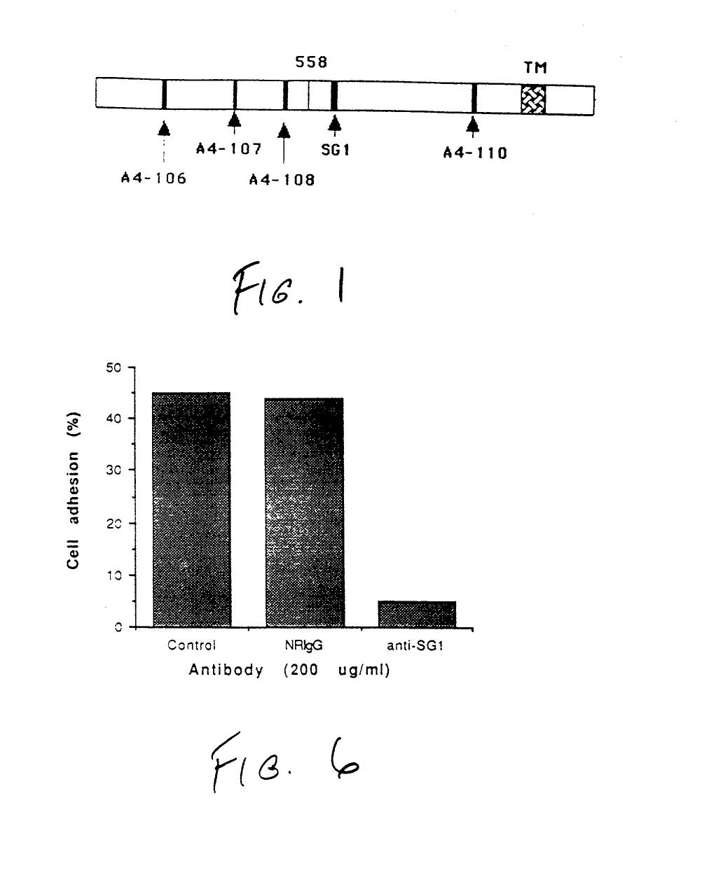

Several synthetic polypeptides derived from the α4 integrin subunit were selected based on positive net charge and negative net hydropathy indices as selection criteria. The synthetic sequences of the polypeptides used in this study are shown in Table I. The polypeptides were synthesized via solid phase synthesis as described herein. Residue numbers were shown according to the previous report by Takada et al., EMBO. J. 8, 1361-1368 (1989). The net charge is calculated from the sum of all charged residues at neutral pH, where K (Lys) and R (Arg) residues are positively charged (+1), E (Glu) and D (Asp) are negatively charged (−1), and histidine (His) is assumed to be uncharged. Hydropathy indexes were calculated by the method of Kyte et al., J. Mol. Biol., 157, 105-132 (1982). Using this method, hydrophilic regions of a protein have negative hydropathy index. The approximate localization of these polypeptides in the α4 integrin subunit is shown in FIG. 1.

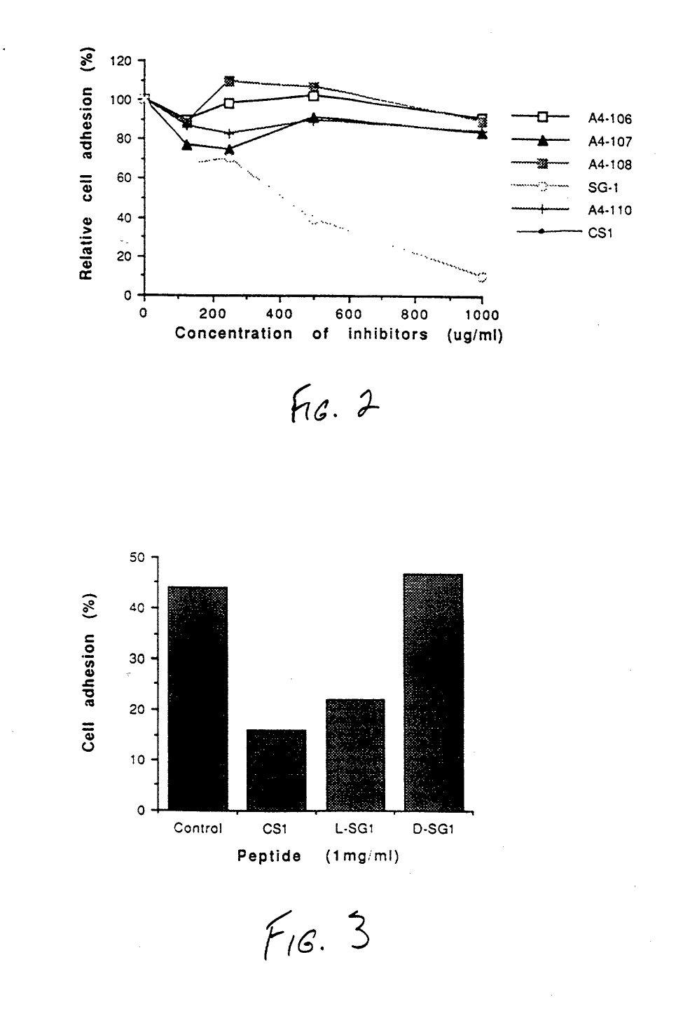

In the first sets of experiments, each of the polypeptides were tested for their abilities to inhibit cell adhesion to substrata coated with CS1/OVA. Cells were preincubated with each peptide for 15 min at 37° C. and then incubated on substrata coated with CS1 for 30 min. Cell adhesion to CS1 was inhibited by soluble CS1, as expected (FIG. 2). Although the synthetic polypeptides tested had similar positive charge and hydropathic index, only the SG1 polypeptide inhibited cell adhesion in a concentration-dependent manner (FIG. 2). The inhibitory effect of CS1 and SG1 was not be due to their toxic activity on the cells, which was estimated by trypan blue exclusion assays (not shown). The inhibitory effect of SG1 was also not due to blocking of binding sites of CS1 by the soluble SG1, because preincubation of CS1-coated plates with soluble SG1 prior to cell adhesion assays also did not affect cell adhesion (not shown). To demonstrate the specificity of inhibitory effect of SG1, an all D-amino acid analog of the SG1 polypeptide (“D-SG1”) was tested for inhibition of cell adhesion to CS1-coated substrate. Plates were coated as described above. Cells were preincubated with 1 mg/ml of the polypeptides for 15 min and then cultured on the substrata coated with CS1/OVA for 30 min. Although both CS1 and L-SG1 significantly inhibited cell adhesion to CS1, D-SG1 did not affect cell adhesion, suggesting that the inhibitory effect of SG1 was specific in nature and dependent upon the chirality of the peptide (see FIG. 3).

### Example 2

Anti-SG1 IgG Inhibited Melanoma Cell Adhesion to CS1

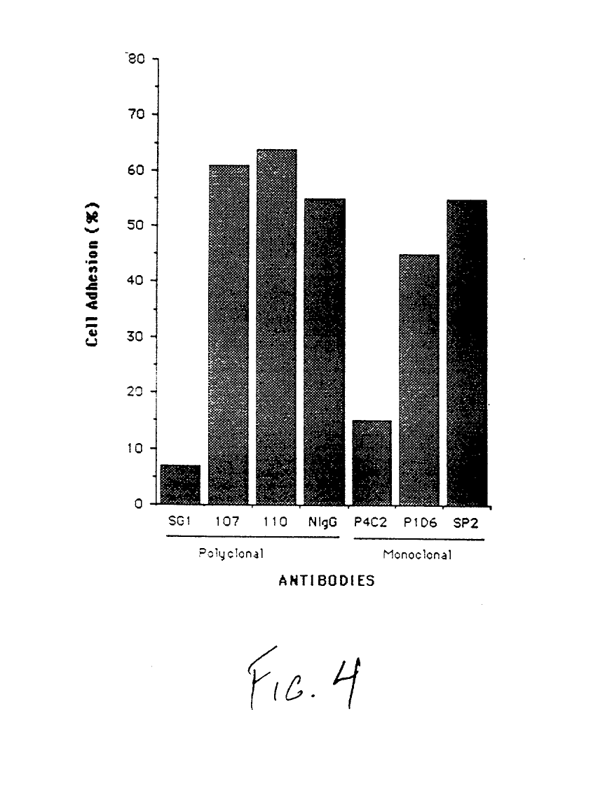

Polyclonal antibodies against the SG1 polypeptide were generated and tested for inhibition of melanoma cell adhesion. As negative controls, polyclonal antibodies against A4-107 and A4-110 were also generated. Plates were coated with 5 μg/ml of CS1/OVA and blocked as described herein. Cells were preincubated with 100 μg/ml of antibodies anti-SG1 IgG, anti-A4-107 IgG, or anti-A4-110 IgG for 15 min at room temperature prior to cell adhesion to substrata coated with 5 μg/ml CS1, which promoted half maximal cell adhesion. Culture supernatants of P4C2, P1D6, and SP2 was wiluted 1:1. Cell adhesion assays were performed for 30 min. Standard error (S.E.) was less than 5%. mAbs Anti-α4 but not anti-α5 inhibited cell adhesion to CS1 (FIG. 4) as expected. Anti-SG1 IgG also inhibited cell adhesion to CS1, however, neither anti-A4-107 nor anti-A4-110 polyclonal antibodies affected cell adhesion (FIG. 4). This demonstrates that the SG1 site plays a specific role in α4β1 integrin-mediated cell adhesion to CS1.

To avoid the possibility of the involvement of Fc portion of anti-SG1 IgG in the adhesion assays, Fab fragments of anti-SG1 IgG were generated as described above and tested for the ability to inhibit cell adhesion to CS1. Cells were preincubated with the indicated concentrations of anti-SG1 IgG, Fab fragments of anti-SG1 IgG, normal rabbit IgG, or Fab fragments of normal rabbit IgG, for 15 min. Cell adhesion assays were performed for 30 min. Standard error (S.E.) was less than 5%.

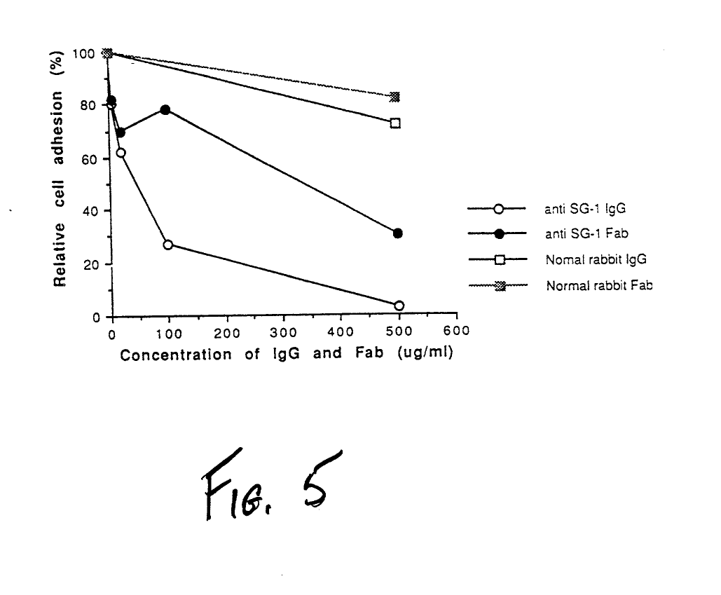

Fab fragments of anti-SG1 IgG also inhibited cell adhesion to CS1 in a concentration-dependent manner (FIG. 5), suggesting that the inhibitory effect of anti-SG1 IgG is of specific nature. Plates were coated with 5 ug/ml of CS1/OVA and blocked as described in Materials and Methods. Cells were preincubated for 15 min. with 100 ug/ml of anti-SG1 IgG or normal rabbit IgG. Cell adhesion assays were performed for 30 min. Standard error (S.E.) was less than 5%. The inhibitory effect of anti-SG1 IgG was not due to the blocking effect of the IgG to chondroitin sulfate since anti-SG1 IgG did not cross react with these ligands determined by ELISA (not shown).

To further demonstrate the specificity of anti-SGI IgG, anti-SG1 IgG was tested for inhibition of cell adhesion to FN-C/H-III and 75 kD fragments. Plates were coated with the respective ligand and blocked as described herein. Cells were preincubated with 100 ug/ml of normal rabbit IgG or anti-SG1 for 15 min. Cell adhesion assays were performed for 30 min. Standard error (S.E.) was less than 5%. Cell adhesion to FN-C/H-III has been shown to be mediated by chondroitin sulfate proteoglycan whereas these cells utilize oc5β1 integrin for binding to the 75 kD fragment. Cell adhesion to CS1 was highly inhibited by anti-SG1 IgG, however, cell adhesion to FN-C/H-III was essentially unaffected (see Table II). Furthermore, anti-SG1 IgG did not affect cell adhesion to the 75 kD fragment (Table II), demonstrating that anti-SG1 IgG specifically inhibits α4β1 integrin function.

Anti-SG1 IgG not only inhibited melanoma cell adhesion, but it was also active against Ramos cell adhesion to a CS1-coated substrate. Ramos cells express predominantly α4β1 integrin and very low levels of α5β1 integrin assessed by flow cytometry (not shown). Ramos cell adhesion to CS1 was significantly inhibited by anti-SG1 IgG (FIG. 6). Ramos cell lysates were immunoprecipitated with P4C2, P5D2, or a control mouse IgG. The immunoprecipitates were separated on SDS-PAGE under reducing condition. The proteins were transferred onto Immobilon-P membrane and incubated with biotinylated anti-SG1 IgG then incubated with peroxidase-conjugated sheep anti-rabbit IgG. The protein was visualized by ECL systems as described above (results not shown).

Anti-SG1 IgG specifically recognized a 150 kD protein in anti-α4 and anti-β1 immunoprecipitates, whereas the protein was not detected in the immunoprecipitates with control mouse IgG. These results suggested that anti-SG1 IgG recognized α4 integrin. Both anti-A4107 IgG and anti-A41 10 IgG also recognized α4 integrin subunit in these experiments.

### Example 3

MPG/NG2 Proteoglycan Directly Interacts with SG1.

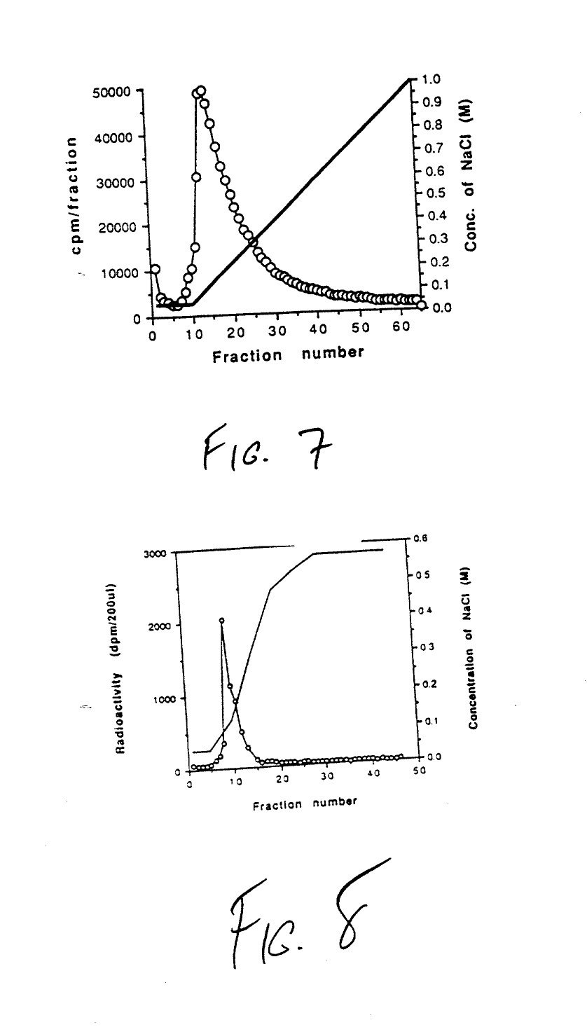

In order to study the function of SG1 site in α4β1 integrin, affinity chromatography was performed using immobilized SG1 polypeptide. Polypeptide SG1 was immobilized on CH-affinity beads as described above. Detergent lysates from ¹²⁵I-labeled melanoma cells were precleared and then applied on the polypeptide column. After extensively washing the columns, the specifically bound proteins were eluted by a linear gradient of NaCl (FIG. 7). Open circles represent the radioactivity in each fraction and the solid line represents the concentration of NaCl in each fraction. The peak fractions were collected and immunoprecipitated with mAbs anti-MPG/NG2 or anti-CD44. The immunoprecipitated proteoglycans were then treated with chondroitinase ABC or heparitinase.

Almost all radioactivity (over 97%) was eluted at 0.13 to 0.15M NaCl. These fractions were pooled and immunoprecipitated with a specific mAbs anti-MPG/NG2 (9.2.27) or anti-CD44. While melanoma cells express chondroitin sulfate in both a modified and unmodified form, the chondroitin sulfate-modified MPG/NG2 was mainly precipitated from the column eluates. CD44 proteoglyean was not detected in the pooled fractions.

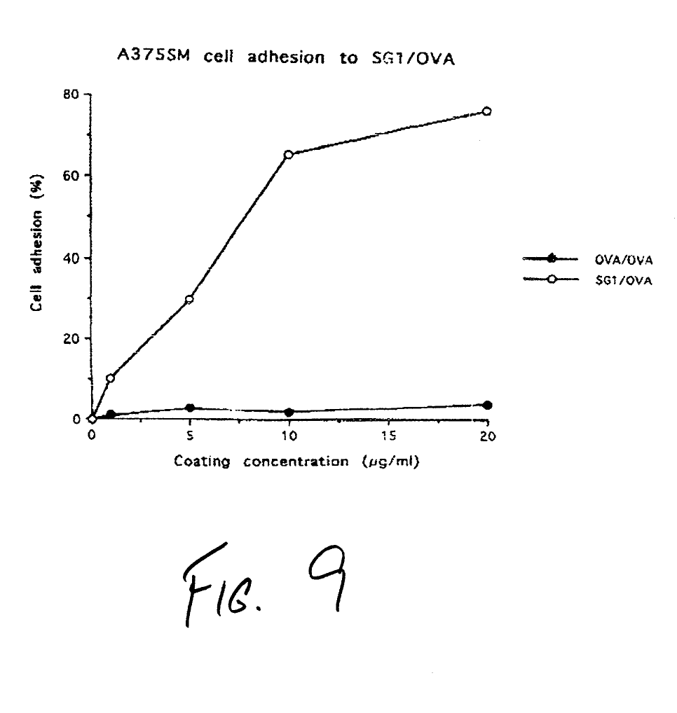

To further demonstrate the nature of interaction between MPG/NG2 and the SG1 polypeptide, the MPG/NG2 chondroitin sulfate protoglycan was purified according to the methods described previously from A375SM melanoma cells and applied on the polypeptide column. MPG/NG2 was eluted by a linear gradient of NaCl at 0.13 to 0.15M NaCl, suggesting the direct interaction between MPG/NG2 and polypeptide SG1 (FIG. 8). As in FIG. 7, open circles represent the radioactivity in each fraction and the solid line represents the concentration of NaCl in each fraction. When purified MPG/NG2 chondroitin sulfate protoglycan was treated with chondroitinase ABC, the MPG/NG2 chondroitin sulfate protoglycan was no longer retained on the SG-1 polypeptide column (not shown), demonstrating that the interaction with the SG1 sequence is mediated through chondroitin sulfate glycosaminoglycan.

### Example 4

Cell Adhesion to an SG1-coated Substrate.

The ability of the SG1 polypeptide to promote the adhesion of a highly metastatic human melanoma cell line A375SM (provided by Dr. I. J. Fidler of Anderson Hospital, University of Texas Health Sciences Center, Houston, Tex.) was examined. The A375SM cell line was maintained in Dulbecco's Modified Eagle's Medium (DMEM) with added vitamins and 10% FBS. The background cell adhesion obtained surfaces coated with ovalbumin (generally <5%) was used as a negative control.

The ability of the A375SM cell line to adhere on the coated surfaces was examined using the following procedure. Flasks of cells were labeled with 30 micro Curies ³H-thymidine (30 microliters) and allowed to incubate overnight at 37° C. Solutions having a range of SG1/ovalbumin concentrations (0.1 to 20 μg/mL) are made up in PBS. Aliquots (50 μl) of each solution were added to the appropriate well of an Immulon 1 (flat bottom) microtiter plate. Replicates of each polypeptide were added to different wells of the microtiter plate which was then allowed to incubate overnight at 37° C.

The next day the flask was visually checked to make sure nothing contaminated the flask and a fresh blocking solution (2.0 mg/mL of bovine serum albumin (BSA) in 1×PBS) was made. The polypeptide conjugate solutions were removed from the Immulon plate by inverting the plate over a sink. Subsequently, 200 microliter aliquots of the blocking solution were added to the plate and allowed to incubate for 2 hours at 37° C. The addition of the blocking solution minimized non-specific adhesion of cells.

Approximately 1.5 hours after blocking the Immulon plate, labeled A375SM celss were harvested by centrifugation (1,000 rpm for 5 min) in PBS containing 1 mM EDTA and washed three times with E.MEM containing 2 mg/ml of BSA. The cells were spun a second time at 1000 rpm, the liquid above the pellet removed and the pellet again resuspended with the serum-free medium. A cell count was taken by placing a sample in a hemocytometer. The cell suspension was then diluted to a final cell density of approximately 100,000 cells per mL in serum-free medium.

The blocking solution was then removed from the peptide-coated Immulon plate by inverting the plate over a sink. Next, 100 microliter aliquots of the cell suspension were added to each well (i.e., about 10,000 cells per well) and the plate was placed in a humidified CO₂ incubator for 30 minutes. After 30 minutes had passed the plate was viewed under the microscope to visually determine if cells had bound in the well. The plate was then washed by adding 100 microliters of serum-free medium, E.MEM containing 2.0 mg/mL of BSA, to each well and subsequently aspirating the solution from each well. Three more washes using 200 microliters of serum-free medium were added to each well and the solution remaining in wells was removed by aspiration. The cells were then lysed by adding 100 microliters of lysis buffer, 0.5 Molar NaOH and 1% SDS, to each well and allowing the plate to incubate for 30 minutes.

The solutions of lysed cells were transferred to 7 mL scintillation vials, starting with the most dilute concentration and ending with the wells coated with the highest concentration of each polypeptide conjugate. One hundred microliters of H₂O and 5 mL of scintillation cocktail were added to each vial and the vials were sealed tightly. The vials were then shaken, placed in the scintillation counter and counted.

The determination of the total amount (100%) of cell associated radioactivity added to each well was carried out by adding 100 microliter aliquots of the cell suspension to each of three 7 mL scintillation vials. To each scintillation vial, 100 microliters of lysis buffer, 0.5 Molar NaOH and 1% SDS were then added and the mixture was allowed to stand for approximately 30 minutes before being placed in the scintillation counter.

The percentage of cells adhering under experimental conditions was calculated according to the formula:

$$100 \times \frac{OBSERVED CPM FOR COATED WELL}{TOTAL CPM OF CELLS ADDED TO EACH WELL} = \begin{matrix}PERCENT CELL ADHESION \\ TO COATED WELLS\end{matrix}$$

The results of the determination of the ability of SG1 polypeptide conjugates to promote the adhesion of A375SM cells is shown in FIG. 9. The SG1/ovalbumin conjugate exhibited strong adhesion promoting activity when the well surfaces were coated with a solution containing 10-20 micrograms/ml of the conjugate.

The publications and patent applications cited in this specification are indicative of the level of ordinary skill in the art to which this invention pertains. All publications and patent applications are herein incorporated by reference to the same extent as if each individual publication or patent application was specifically and individually incorporated by reference.

The invention has been described with reference to various specific and preferred embodiments and techniques. However, it should be understood that many variations and modifications may be made while remaining within the spirit and scope of the invention.

TABLE I
Synthetic Polypeptides Derived From Human α4 Integrin Subunit

| | | Net | Net | Sequence |
|---|---|---|---|---|
| Primary sequence | Residue# | charge | hydropathy | ID No. |
| KYKAFLDKQNQVKFG | 201-215 | +3 | −14.7 | 4 |
| (A4-106) | | | | |
| LHEMKGKKLGS | 261-271 | +2 | −8.8 | 5 |
| (A4-106) | | | | |
| NRKAESPPRFY | 491-501 | +2 | −14.2 | 6 |
| (A4-107) | | | | |
| SSREANCRTHQAFM- | 518-537 | +2 | −20.7 | 7 |
| RKDVRD | | | | |
| (A4-108) | | | | |
| LQQKKEKDIMKKTI | 570-583 | +3 | −14.4 | 2 |
| (SG1) | | | | |
| KKEKDIMKKT | 573-582 | +3 | −14.4 | 3 |
| (SG1a) | | | | |
| SKTDKRLLY | 848-856 | +2 | −6.0 | 8 |
| (A4-110) | | | | |

TABLE II
Anti-SG1 Inhibition of melanoma cell adhesion to
Substrates coated with fibronectin fragments.

| | Cell adhesion (%) | |
|---|---|---|
| Substrata | Normal rabbit IgG | Anti-SG1 IgG |
| CS1 | 55 | 5 (87)^{a} |
| FN-C/H-III | 62 | 58 (6) |
| Fibronectin 75 kD | 60 | 55 (8) |
| fragment | | |

^{a}% of inhibition versus normal rabbit control shown in parentheses.

What is claimed is:

1. A polypeptide having no more than about 50 amino acid residues and comprising an amino acid sequence corresponding to KKEKDIMKK (SEQ ID NO:1) or a variant thereof, said variant having at least 9 amino acid residues and differing from KKEKDIMKK (SEQ ID NO:1) through one or more deletions, conservative amino acid substitutions or combinations thereof.

2. The polypeptide of claim 1 wherein said variant has a net charge of at least +3 at neutral pH.

3. The polypeptide of claim 1 wherein said variant has a net hydropathy of about −10 to about −20.

4. The polypeptide of claim 1 wherein said conservative amino acid substitutions comprise substitution of a positively charged amino acid for a lysine residue.

5. The polypeptide of claim 1 wherein said conservative amino acid substitutions comprise substitution of a negatively charged amino acid for an aspartate or glutamate residue.

6. The polypeptide of claim 1 wherein said conservative amino acid substitutions comprise substitution of an uncharged amino acid for a methionine or isoleucine residue.

7. The polypeptide of claim 1 wherein said deletion comprises deletion of an isoleucine or methionine residue.

8. The polypeptide of claim 1 wherein said polypeptide is capable of modulating cellular adhesion.

9. The polypeptide of claim 1 wherein said polypeptide is capable of inhibiting the adhesion of melanoma cells to CS1-coated substrate.

10. The polypeptide of claim 1 wherein said polypeptide is capable of modulating adhesion of Ramos cells.

11. The polypeptide of claim 1 having the formula
LQQKKEKDIMKKTI (SEQ ID NO:2).

12. The polypeptide of claim 1 having no more than about 25 amino acid residues.

13. A polypeptide/carrier molecule conjugate comprising at least one polypeptide covalently linked to a carrier molecule, wherein the polypeptide has no more than about 50 amino acid residues and comprises an amino acid sequence corresponding to KKEKDIMKK (SEQ ID NO:1) or a variant thereof, said variant having at least 9 amino acid residues and differing from KKEKDIMKK (SEQ ID NO:1) through one or more deletions, conservative amino acid substitutions or combinations thereof.

14. The polypeptide/carrier molecule conjugate of claim 13 wherein the carrier molecule comprises a biological carrier molecule.

15. An affinity purification device comprising a conjugate of a chromatography support and a polypeptide having no more than about 50 amino acid residues and comprising an amino acid sequence corresponding to KKEKDIMKK (SEQ ID NO:1) or a variant thereof, said variant having at least 9 amino acid residues and differing from KKEKDIMKK (SEQ ID NO:1) through one or more deletions, conservative amino acid substitutions or combinations thereof.

16. A method for modulating the adhesion of cells to a substrate comprising:
adding a polypeptide to a suspension of the cells to form a modified cell suspension; and
contacting the modified cell suspension with the substrate;
wherein the polypeptide has no more than about 50 amino acid residues and comprises an amino acid sequence corresponding to KKEKDIMKK (SEQ ID NO:1) or a variant thereof, said variant having at least 9 amino acid residues and differing from KKEKDIMKK (SEQ ID NO:1) through one or more deletions, conservative amino acid substitutions or combinations thereof.

17. A peptide comprising a F_{ab} fragment from an antibody specific for an antigen within an amino acid sequence corresponding to KKEKDIMKK (SEQ ID NO: 1) or a variant thereof, said variant having at least 9 amino acid residues and differing from KKEKDIMKK (SEQ ID NO:1) through one or more deletions, conservative amino acid substitutions or combinations thereof.

18. The peptide of claim 17 wherein the antibody is specific for an antigen within an amino acid sequence corresponding to LQQKKEKDIMKKTI (SEQ ID NO:2).

19. The peptide of claim 18 wherein said peptide is an IgG antibody specific for the antigen.

20. An amphiphilic molecule comprising a polypeptide fragment which has no more than about 50 amino acid residues and includes an amino acid sequence corresponding to KKEKDIMKK (SEQ ID NO:1) or a variant thereof, said variant having at least 9 amino acid residues and differing from KKEKDIMKK (SEQ ID NO:1) through one or more deletions, conservative amino acid substitutions or combinations thereof.

21. A chemodiagnostic device for detection of a cell growth comprising a polypeptide having no more than about 50 amino acid residues and including an amino acid sequence corresponding to KKEKDIMKK (SEQ ID NO:1) or a variant thereof, said variant having at least 9 amino acid residues and differing from KKEKDIMKK (SEQ ID NO:1) through one or more deletions, conservative amino acid substitutions or combinations thereof,
wherein said polypeptide modulates the adhesion of cells from the cell growth.

22. A prosthetic device designed for placement in vivo comprising a surface coated with a composition which includes a polypeptide having no more than about 50 amino acid residues and including an amino acid sequence corresponding to KKEKDIMKK (SEQ ID NO:1) or a variant thereof, said variant having at least 9 amino acid residues and differing from KKEKDIMKK (SEQ ID NO:1) through one or more deletions, conservative amino acid substitutions or combinations thereof,
wherein said polypeptide modulates cellular adhesion.

23. A cell culture substrate comprising a surface coated with a composition which includes a polypeptide having no more than about 50 amino acid residues and including an amino acid sequence corresponding to KKEKDIMKK (SEQ ID NO:1) or a variant thereof, said variant having at least 9 amino acid residues and differing from KKEKDIMKK (SEQ ID NO:1) through one or more deletions, conservative amino acid substitutions or combinations thereof,
wherein said polypeptide modulates cellular adhesion.

24. A percutaneous device designed for placement in vivo comprising a surface coated with a composition which includes a polypeptide having no more than about 50 amino acid residues and including an amino acid sequence corresponding to KKEKDIMKK (SEQ ID NO:1) or a variant thereof, said variant having at least 9 amino acid residues and differing from KKEKDIMKK (SEQ ID NO:1) through one or more deletions, conservative amino acid substitutions or combinations thereof.
